(19) United States
(12) Patent Application Publication
(10) Pub. No.: US 2026/0341139 A1
(43) Pub. Date: Oct. 29, 2026

(54) Door Monitoring System With Serial Communications Protocol

(71) Applicant: Kingsway Enterprises (UK) Limited, Sidcup Kent (GB)

(72) Inventors: Julian Hall, Sidcup Kent (GB); Rod Foot, Sidcup Kent (GB); Peter Deeming, Sidcup Kent (GB)

(21) Appl. No.: 19/699,333

(22) Filed: Jun. 5, 2026

Related U.S. Application Data
(63) Continuation of application No. PCT/GB2024/053034, filed on Dec. 4, 2024.

(30) Foreign Application Priority Data
Dec. 7, 2023 (GB) 2318724.8

(51) Int. Cl. G08B 21/04; G01L 5/00; G16H 40/67; H04L 67/12

(52) U.S. Cl. CPC G08B 21/0423; G01L 5/0052; G08B 21/0469; G16H 40/67; H04L 67/12

(57) ABSTRACT

A computer-implemented method for performing by a door monitoring system is disclosed. The method comprises monitoring a door, the door comprising one or more patient safety devices and an embedded electronics module configured to monitor a status of each patient safety device. The method further comprises receiving status data from the embedded electronics module via a hardwired serial communications protocol and determining, based on the received status data, that a status of one of the one or more patient safety devices has changed. The method further comprises recording the status change.

### RELATED APPLICATION

The present application is a continuation of International Application No. PCT/GB2024/0053034, filed Dec. 4, 2024, which claims priority to Great Britain Patent Application No. 2318724.8 filed Dec. 7, 2023. The above‑referenced applications are hereby incorporated by reference into the present application in their entirety.

### TECHNICAL FIELD

The present disclosure relates to methods of implementing a door monitoring system, in particular in settings where the doors contain patient safety devices configured to mitigate self-harm or aid patient welfare within a healthcare facility.

### BACKGROUND

In psychiatric hospitals, a problem exists that patients may attempt to cause themselves harm in a variety of ways. One common method of attempting self-harm is by using a ligature created by securing a rope or cable around an available anchor point in a room. One solution to this problem is to design room fixtures and fittings such that they do not provide such anchor points. However, in some cases this is difficult or impossible. An example of this is door fittings. Individuals may try to create a ligature by securing a rope or cable around an edge of a door leaf.

One solution is to attach a ligature-detection device, typically a switch, to an edge (e.g. top edge) of a door leaf. When a ligature is secured around the device and pressure is applied, the switch is caused to close, thereby completing or breaking an electrical circuit and activating an alarm. Accordingly, while the door leaf may itself remain a potential ligature hazard, safety is nonetheless improved because a nearby healthcare professional is alerted when an individual attempts to secure a ligature around the door leaf. Other patient safety devices may be used to monitor other aspects of patient welfare in a similar manner.

Unfortunately, problems in preventing self-harm in this manner remain. In particular, it may take staff a long time to determine the source of an alarm and find the correct room so that they may administer assistance. Additionally, patient safety devices are vulnerable to tampering and signal loss. There is often a significant delay between a patient safety device being tampered with or rendered ineffectual and staff becoming aware of this. This ‘down-time’ in the patient safety device undermines the safety of patients.

Further issues arise as a result of patient safety devices and alarm systems being administered by different parties. Often, different third parties operate different aspects of a door monitoring system, such as monitoring patient safety devices, sounding alarms, and generating recording of event data. This requires multiple third party systems to interface, which can lead to reliability issues as well as confusion for staff members who have to interact with multiple systems.

As can be seen, existing mechanisms for monitoring doors comprising patient safety devices suffer from significant drawbacks. It would be advantageous to provide systems and methods which address one or more of these problems, in isolation or in combination.

### Overview

This overview introduces concepts that are described in more detail in the detailed description. It should not be used to identify essential features of the claimed subject matter, nor to limit the scope of the claimed subject matter.

According to a first aspect of the present disclosure, a computer-implemented method for performing by a door monitoring system is disclosed. The method comprises monitoring a door. Monitoring the door may comprise, for example, configuring a controller of the door monitoring system to receive and record data relating to a status of one or more devices associated with the door. In this context, a door may be considered as comprising both the door leaf and the door frame. Accordingly, any references herein to devices or components comprised by a door are to be interpreted as including the possibility of the devices or components being comprised by the door leaf and/or by the door frame.

According to the present disclosure, the monitored door comprises one or more patient safety devices. In this context, a “patient safety device” can be considered as encompassing any device included in a door (whether in the door leaf or door frame) that is configured to aid patient safety or wellbeing, particularly in terms of mitigating self-harm. Examples of patient safety devices include a ligature-detection device, configured to detect whether a ligature is being attached to the door (typically to the door leaf). Other examples of patient safety devices include a door observation panel, which is a panel included in a door leaf that can be opened from the outside by a staff member to enable the staff member to see into a patient’s room and thereby check on the wellbeing of the patient. Other examples of patient safety devices include a shock sensor, configured to detect shocks impacting the door. These shocks can be indicative of a patient striking a door, which could be a sign of self-harm and/or aggression which needs to be attended to. Other examples of patient safety devices include an anti-barricade switch which, once triggered, enables some or all of the door to be opened outwards. This enables staff to access a patient’s room even if the patient has barricaded the door from the inside. Another example of a patient safety device is a door-open monitor, configured to record when a door is opened and/or closed. Other potential patient safety devices that aid patient safety and/or mitigate self-harm will be apparent to a skilled reader. That statuses of these and other devices can be monitored by the door monitoring system of the present disclosure.

According to the present disclosure, the monitored door further comprises an embedded electronics module configured to monitor a status of each patient safety device comprised in the monitored door. An “embedded electronics module” in this context can be considered as an electrical module that is comprised by the door (whether the door leaf or door frame) and is configured to monitor patient safety devices of the door. In particular, the embedded electronics module is configured to receive data from patient safety devices that are comprised by the door and send this data on to a controller of the door monitoring system. As a result, the embedded electronics module advantageously acts as a “one stop shop” for the door monitoring system to obtain data about the monitored door. In other words, status data for a plurality, and potentially all, patient safety devices in the monitored door can be obtained by the door monitoring system from this one single embedded electronics module. Accordingly, the controller of the door monitoring system need only be connected to the embedded electronics module rather than requiring parallel connections from the controller to every patient safety device comprised in the door. This simplified architecture is highly advantageous in settings where doors comprise multiple patient safety devices. The present inventors have identified that in such scenarios parallel connections between the controller and each patient safety device quickly results in an unpractical and inefficient amount of wiring and hardwire. Incorporation of the embedded electronics module addresses this problem.

The disclosed method further comprises receiving, by the door monitoring system, status data from the embedded electronics module via a hardwired serial communications protocol. The method then comprises determining, based on the received status data, that a status of (at least) one of the one or more patient safety devices has changed. As already noted, status data for a plurality of patient safety devices in the door can thereby be obtained via this one, single serial communications protocol, preferably via a single serial communications protocol output of the embedded electronics module. This arrangement significantly simplifies the obtaining of data from patient safety devices of the monitored door, as already noted.

The method further comprises recording, by the door monitoring system, the status change(s) of the patient safety device(s). For example, a controller of the door monitoring system may record status change data in a database such as a database stored and/or operated by the controller, which can be a local database or a server or cloud-based database. By recording status changes of patient safety devices, an accurate event log can be built up. Such event logs are important, for example, to enable effective auditing of healthcare institutions to ensure that patient welfare is being maintained and to ensure that any events that could impact patient safety are being recorded.

As noted above, the one or more patient safety devices may comprise a ligature-detection device. In that case, determining that a status of the ligature-detection device has changed may comprise determining that the ligature-detection device has been triggered, as may happen when a patient attempts to secure a ligature to the door. Recording such events accurately and effectively is important to ensure that staff members can respond promptly to provide assistance to the patient.

As also noted above, the one or more patient safety devices may comprise a door observation panel. In that case, determining that a status of the door observation panel has changed may comprise determining that the door observation panel has been opened, as may happen when a staff member looks into a patient’s room via the door observation panel. Recording such events accurately and effectively is important to enable the regularity with which patients are being checked on to be recorded accurately and monitored. This is turn is important for ensuring patient wellbeing and ensuring that patients are not being neglected.

As also noted above, the one or more patient safety devices may comprise a shock sensor. In that case determining that a status of the shock sensor has changed may comprise determining that the shock sensor has recorded a shock impacting on the monitored door, as may happen when a patient strikes (e.g. hits, kicks or otherwise impacts) a door. These events can be symptomatic of self-harm and/or aggression, and recording such events effectively and accurately is important to enable staff to quickly render assistance or prevention in a suitable manner.

As also noted above, the one or more patient safety devices may comprise an anti-barricade switch. In that case, determining that a status of the anti-barricade switch has changed may comprise determining that the anti-barricade switch has been triggered. Triggering of anti-barricade switches is typically associated with a high-risk event where a patient has barricaded a door. Accordingly, recording these events is important for enabling other staff members to realise that a door-barricading event is occurring and administer additional assistance to the staff member who triggered the anti-barricade switch. Accidental triggering of the anti-barricade switch may also present a safety risk for patients, which is another reason why such events need to be accurately recorded.

As also noted above, the one or more patient safety devices may comprise a door-open monitor. In that case, determining that a status of the door-open monitor has changed may comprise determining that the door has been opened. Accurately monitoring when patients’ doors are opened is important for ensuring patient safety and understanding patient whereabouts.

The method may further comprise determining, based on the received status data, that a fault with one of the one or more patient safety devices has been detected. It will be appreciated that a fault with any patient safety device can present a safety risk to patients. Accurately recording such events is therefore important to ensure that the faults can be rapidly responded to and repaired.

The method may further comprise determining, based on the received status data, that the status of one of the one or more patient safety devices can no longer be accurately determined. Such an event may occur if a device, or the connection between a monitoring component and the device, is defective and/or has been tampered with. Accurately recording such events is again important to enable rapid response and ensure patient safety.

The method may further comprise determining, based on the received status data, that one of the one or more patient safety devices has not responded to a status update request. In some implementations, the controller of the door monitoring system may send, to the embedded electronics module, status update requests for the statuses of the patient safety devices of the door. Failure to respond to such a request, whether by the patient safety device or the embedded electronics module, may again be indicative that a part of the monitoring system, or a connection thereto is defective or has been tampered with. Once again, accurately recording such events is important to enable rapid response and ensure patient safety.

The method may further comprise displaying a floor plan of a building comprising the monitored door. For example, a floor plan of the building may be displayed on a display screen connected to the controller of the door monitoring system. Responsive to determining, based on the received status data, that a status of one of the one or more patient safety devices has changed, the method may further comprise indicating that a status of the patient safety device has changed on the displayed floor plan. For example this may be achieved by modifying, on the displayed floor plan, the appearance of the monitored door containing the device that has recorded a status change. Additionally or alternatively, the appearance on the displayed floor plan of an area around, adjacent to or associated with the monitored door containing the device that has recorded a status change may be modified. For example, the appearance of the room to which the door in question leads may be modified on the floor plan.

Such a method provides an intuitive and clear mechanism for visually alerting staff members to where a status change of a patient safety device has occurred. In particular, staff members are provided with the geographical context of a status change because the door in question is shown on a displayed floor plan and the appearance of the door is modified to make it stand out from other doors. This allows staff members to locate the required door and provide assistance to that location more quickly and easily, improving response times and patient safety.

It will be appreciated that modifying the appearance of the monitored door on the displayed floor plan may be performed in any suitable manner, with the aim of drawing attention to the door on the display. The modification may be made to the door itself on the display, and/or to a visual indicator or marker associated with (e.g. provided near or overlaid on) the door on the floor plan.

In this manner, changes in the statuses of patient safety devices associated with doors can be intuitively displayed to a user, such as a psychiatric nurse. As will be appreciated, a variety of modifications in how the door is displayed on the floor plan are envisaged. For example, the door may be visually emphasised, such as through the door or an associated icon beginning to flash. Alternatively or additionally, the door or an associated icon may change colour. The door may also be added to a displayed list of doors associated with the change of status that has taken place. For example, if the ligature-detection device of a door has been triggered, an icon overlaid on the door on the displayed floor plan may begin to flash and the door may appear on an alert list of doors provided on the display.

The modification of the appearance of the monitored door may differ based on which patient safety device has recorded a status change. For example, triggering of a ligature-detection device of a door may result in a different visual representation of the door on the displayed floor plan compared with a door observation panel being opened. It will be appreciated that it can be beneficial if certain more high risk events (such as ligature-detection events) are associated with more distinctive or noticeable changes in appearance on the displayed floor plan, so as to better convey the severity of the event to staff members.

As noted above, alternatively or additionally to modifying the appearance of the monitored door itself in response to a status change, the disclosed method may comprise modifying, on the displayed floor plan, the appearance of a room or area adjacent to or associated with the monitored door where the status change has occurred. For example, the appearance of the room to which the door leads (such as a patient’s bedroom) may be modified on the displayed floor plan. All of the functionality relating to modifying the appearance of the displayed door described above and below applies equally to modifications made to the appearance of the room or area adjacent or associated with the door on the displayed floor plan. In particular, any suitable form of visual modification can be made to the room or area, or to an icon associated therewith. Such modifications are described in further detail below in the context of modifying the appearance of a door but apply equally to modifying the appearance of a displayed room or area.

Responsive to determining, based on the received status data, that a status of one of the one or more patient safety devices has changed, the method may further comprise causing an auditory indication to be sounded. For example, an alarm or alert may be sounded at one or more locations around the building, such as via a loudspeaker or alarm system.

Auditory indications (alarms) may complement use of a display to show where an alarm-causing event has taken place. For example, an auditory indication may be particularly useful when a display is not under constant surveillance. For example, a display may be provided in a hallway in a psychiatric ward. When the status of a patient safety device changes, an auditory alert or alarm may be sounded, thereby drawing staff members’ attention to the display where the location of the status change is being displayed. This helps staff members locate status changes and administer assistance quickly.

The auditory indication may differ based on which patient safety device has recorded a change. Accordingly, for example, a ligature-detection event may result in a louder, more persistent or more distinctive or unusual sound being played than a door opening/closing event or a fault detection event. In some examples, an auditory indication is only used when a ligature-detection device is triggered, given the urgency of such an event.

If a status of a patient safety device changes, the method may comprise determining a duration for which the status change has persisted. In other words, the method may comprise determining how long the status change has lasted. The nature of any auditory alerts or visual modifications can then be based on this determined duration.

For example, the method may comprise providing an auditory indication that the status of a patient safety device has changed, wherein the auditory indication is based on the duration for which the status change has persisted. In one example, the auditory indication may be configured such that: when the status change has persisted for less than a threshold duration, a first type of auditory indication is sounded; and when the status change has persisted for more than a threshold duration, a second (different) type of auditory indication is sounded.

In one example, if the status change has persisted for less than a given threshold duration, for example seven seconds, then a first type of auditory alarm is sounded. The first type of auditory alarm may be relatively short in duration, may have a relatively low volume and may have a distinct tone indicative of a brief triggering of the ligature-detection device. Such an indication may be considered as a “pre-alarm”. An example of a pre-alarm may be a single, low-volume tone that is sounded once.

If the status change persists (i.e. continues to be detected) beyond the threshold duration, then a second type of auditory alarm may be sounded. The second auditory alarm may be relatively long in duration (or be continuous/constant), may have a relatively high volume and may have a distinct tone indicative of a prolonged triggering of the device. Such an indication may be considered as a “full-alarm”. An example of a full alarm may be a high-volume tone that is sounded continuously.

This functionality is advantageous in that a brief status change at a patient safety device, which may be benign, can be distinguished easily and quickly from a prolonged status change, which is more likely to represent a high risk event. It will be apparent that a variety of tonal effects may be applied to ensure that a full-alarm is perceived as more urgent than a pre-alarm and is more likely to catch the attention of a member of staff at the facility.

Pre-alarms and full-alarms can also be distinguished visually. In particular, the manner in which the monitored door is presented on the displayed floor plan may be modified based on the duration for which the status change has persisted.

In one example, the manner in which the selected door is presented on the displayed floor plan is modified such that: when the status change has persisted for less than a threshold duration, a first type of visual modification is applied; and when the status change has persisted for more than a threshold duration, a second (different) type of visual modification is applied.

In other words, the pre-alarm and full-alarm functionality described above in the context of an auditory indication may be implemented also (or alternatively) with visual equivalents. Accordingly, in some examples, when the threshold duration has not yet been exceeded, a first type of visual modification may be applied to the door in question. For example, the door (or an icon associated with it) may flash (or pulse) slowly or may change colour to a first colour (e.g. orange). If the threshold duration is exceeded, a second type of visual modification may be applied. In particular, the nature of the visual modification may change to become more eye-catching and to convey more easily the severity of the situation. For example, the door (or an icon associated with it) may flash (or pulse) rapidly or may change colour to a second colour (e.g. red). As in the case of the auditory indications mentioned above, such visual indicators can quickly and effectively convey the severity of a status change at a patient safety device.

For both auditory and visual indicators, the threshold duration between the “pre-alarm” and “post-alarm” functionality may be at least 2 seconds. The present inventors have identified that setting the threshold duration at or above this duration minimises the number of false alarms, in other words full-alarms that are caused by accidental or benign triggering of patient safety devices. This is because benign or accidental triggering events typically last less than 2 seconds.

Similarly, the threshold duration between the “pre-alarm” and “post-alarm” functionality may be at most 7 seconds. The present inventors have identified that setting the threshold duration at or below this duration ensures that actual status changes at patient safety devices result in a full alarm sufficiently quickly to enable a swift enough response. If the threshold is set higher than 7 seconds, then staff will be delayed to a potentially dangerous extent in responding to status changes, which can be particularly problematic if the status change represents a ligature attachment event.

Hence, the threshold duration is advantageously between 2 and 7 seconds. For example, the threshold duration may be 2 seconds, 3 seconds, 4 seconds, 5 seconds, 6 seconds or 7 seconds.

By providing a single door monitoring system that provides each of the event detection, event logging and alarm/display-related mechanisms described above, the reliability and accessibility of the system is improved compared to existing mechanisms where all three of these aspects are provided by different parties and distinct systems. In particular, there is no need for individual third party systems to interface or work together, minimising risks of systems clashing or not interfacing correctly. Because everything is controlled via a single unitary system, it is also no longer necessary for staff members to understand and interact with multiple disparate systems. That said, the disclosed system of course does not preclude the use of third party systems or devices which can be incorporated into the network and can be monitored by the door monitoring system.

It will be appreciated that the status data for patient safety devices described above may be obtained in any suitable way. In one example, the method may comprise periodically polling, by the controller of the door monitoring system, the embedded electronics module with a request for updated status data. For example, the controller may request a status update for each patient safety device that a particular embedded electronics module is connected to. This polling of the embedded electronics module may occur at least once every 10 seconds. This regularity of polling ensures that status changes are notified to the controller sufficiently quickly to enable staff to respond in a timely manner. Polling of the embedded electronics module in this manner is also a way to determine that the embedded electronics module is still active and responsive. Hence, the regular polling of the embedded electronics module is a manner of ensuring that faults with the system are identified sufficiently quickly.

Additionally or alternatively to providing status data responsive to a polling request, the embedded electronics module may be configured to automatically transmit any updated status changes it identifies to the controller as soon as it identifies the status change. In this way, the controller is notified immediately of the status change, rather than waiting until it next polls the embedded electronics module.

Similarly to how the controller may poll the embedded electronics module, the embedded electronics module may periodically poll the one or more patient safety devices for an indication of their respective statuses. For example, the embedded electronics module may request a status update from each patient safety device which it is monitoring multiple times per second, for example hundreds of times per second. This regularity of polling ensures that status changes are notified to the embedded electronics module, and by extension the rest of the door monitoring system, sufficiently quickly to enable staff to respond in a timely manner.

In an alternative implementation, status data may be obtained by a patient safety device sending a status-change update message upon recording a status change. These messages can be forwarded via the embedded electronics module to the controller of the door monitoring system. It will be appreciated that a combination of polling and “message upon status change” functionality can also be used.

According to a further aspect of the present disclosure, a computer apparatus configured to perform any of the methods described herein is disclosed. The computer apparatus may, for example, be comprised by a controller of the door monitoring system such that the controller can perform the disclosed methods. Accordingly, according to a further aspect of the present disclosure, a door monitoring system comprising a controller having a processor and memory, the controller configured to carry out any of the methods described herein, is disclosed.

According to a further aspect of the present disclosure, a computer-readable medium comprising instructions which, when executed by a computer, cause the computer to carry out any of the methods described herein is disclosed.

According to a further aspect of the present disclosure, a computer program comprising instructions which, when the program is executed by a computer, cause the computer to carry out any of the methods described herein is disclosed.

According to a further aspect of the present disclosure, a door for monitoring by a door monitoring system is disclosed. The door comprises a door leaf, one or more patient safety devices, and an embedded electronics module configured to monitor a status of each patient safety device. The embedded electronics module is configured to transmit status data for the one or more patient safety devices to a controller of the door monitoring system via a hardwired serial communications protocol. Such a door can be used in the contexts of any of the methods or systems disclosed herein and has the same advantages as discussed above and below in relation to these systems and methods.

Advantageously, the embedded electronics module may be configured to receive input status data from a plurality of patient safety devices of the door. The embedded electronics module may further be configured to provide the status data from the plurality of patient safety devices to the controller of the door monitoring system via a single serial communications protocol output port. The embedded electronics module can in this sense be considered as a form of “many-to-one” concentrator of status signals. This concentration of status data means the data can now be accessed by the controller of the door monitoring system via a single output port of the embedded electronics module. This greatly simplifies the system and methodology by which the door monitoring system obtains the status data for each patient safety device.

The one or more patient safety devices may be comprised in the door leaf, a door frame or a combination of both. Similarly, the embedded electronics module may be comprised in the door leaf or the door frame.

### BRIEF DESCRIPTION OF THE FIGURES

Illustrative implementations of the present disclosure will now be described, by way of example only, with reference to the drawings. In the drawings:

FIG. 1 shows a door monitoring system according to an example implementation of the present disclosure;

FIG. 2 shows an example door that may be monitored by the door monitoring system;

FIG. 3 shows an example embedded electronics module that may be comprised by a door of the present disclosure;

FIG. 4 shows a method of identifying, by a door monitoring system, a change of status at a patient safety device of a monitored door according to the present disclosure;

FIG. 5 shows an example graphical user interface for displaying status changes identified by the door monitoring system; and

FIG. 6 shows an example computer apparatus that can be used to implement the methods of the present disclosure.

Throughout the description and the drawings, like reference numerals refer to like features.

### DETAILED DESCRIPTION

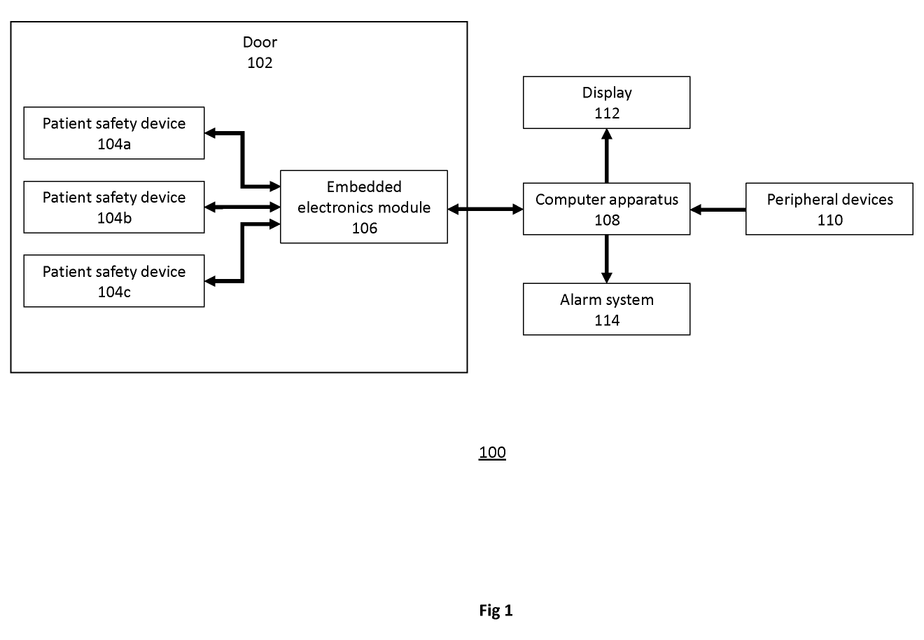

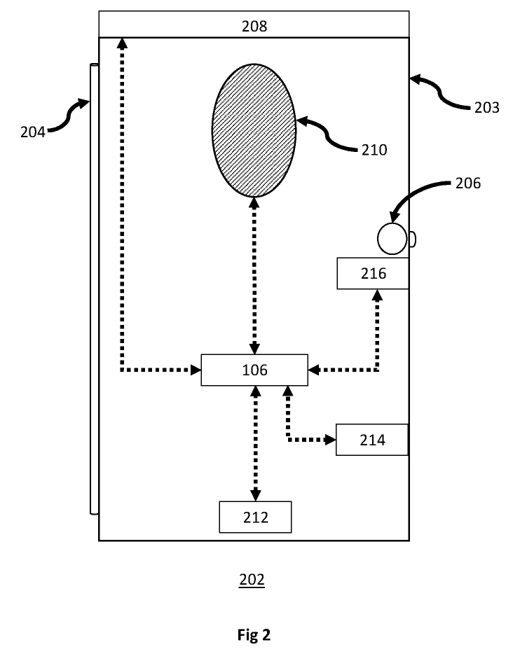

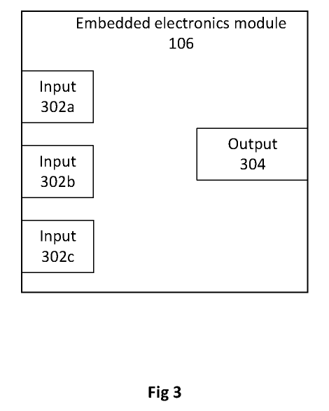

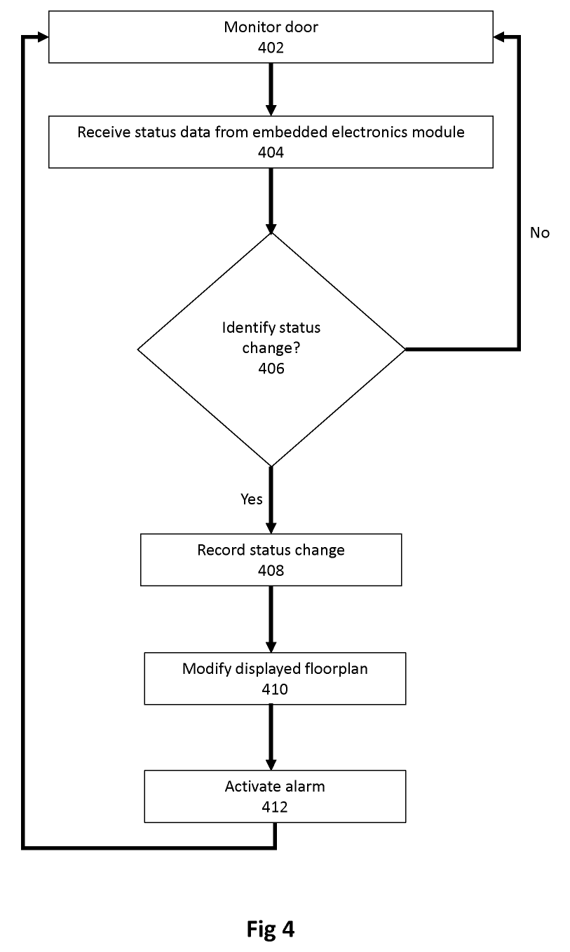

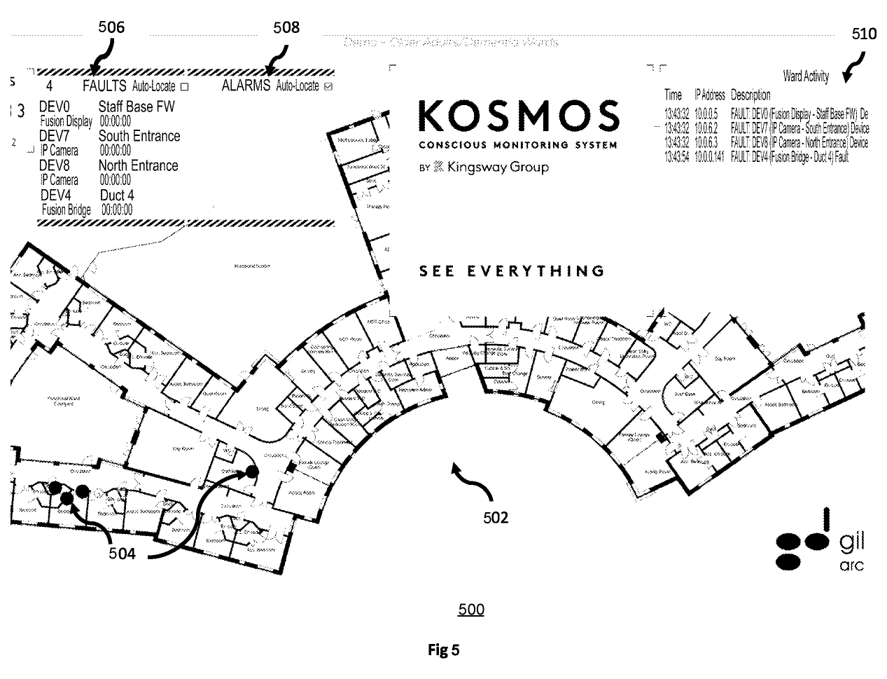

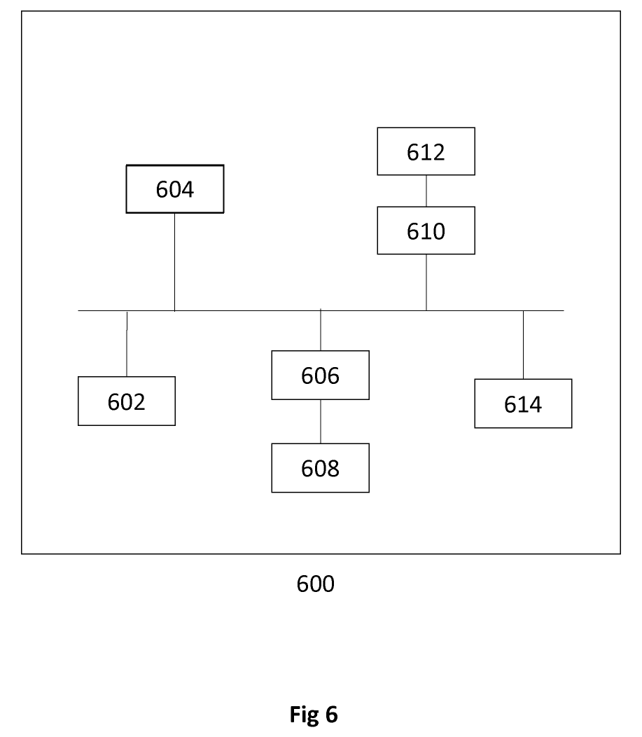

This detailed description describes, with reference to FIG. 1, a door monitoring system that can be used to implement the methods disclosed herein. An example door comprising patient safety devices that can be monitored by the door monitoring system is described with reference to FIG. 2. An embedded electronics module that can be used to transmit status data to a controller of the door monitoring system is described with reference to FIG. 3. Methods of using the disclosed door monitoring system are discussed in relation to FIG. 4. An example graphical user interface which can be used to display indications of identified status changes is described in reference to FIG. 5. Finally, a computer apparatus which may form part of the door monitoring system and may be used to implement the methods disclosed herein is described with reference to FIG. 6.

The methods disclosed herein relate generally to door monitoring systems and methods for configuring and using the same. As noted above, in certain secure environments such as psychiatric hospitals, doors may be configured to contain patient safety devices. These are devices or sensors configured to monitor some aspect of the door or the environment around the door, such as a patient’s room. The status data obtained from these devices is therefore important for ensuring patient safety and wellbeing. The disclosed systems and methods enable such data from a plurality of patient safety devices of a door to be easily monitored and provided to the door monitoring system, via a single embedded electronics module. In particular, patient safety devices according to the present disclosure may advantageously be configured to exhibit a status change responsive to some external input, such as a pressure being applied to a ligature-detection device. In some cases, patient safety devices may simply comprise a switch that either exhibits a binary “open” or “closed” state. A change from one of these states to the other can represent a status change and can be identified by the door monitoring system. In other implementations, patient safety devices may be configured to record a plurality of more nuanced or sophisticated statuses which can be communicated to or obtained by the door monitoring device.

As will be described more fully below, the door monitoring system can take appropriate action in response to the received status data, for example by causing a visual alert and/or an auditory alarm. The door monitoring system can also record the status change data to provide an accurate record of patient-safety related events. The disclosed method and system thereby enable patient safety to be improved through more efficient and reliable recording of data for patient safety devices. The disclosed methods and systems also provide a more intuitive and reliable mechanism for both configuring and using the door monitoring system in an environment containing doors having patient safety devices, as will be described in more detail below.

Turning first to FIG. 1, an example implementation of a door monitoring system 100 is schematically shown. Such a door monitoring system may be used to enable monitoring of doors in a location such as a psychiatric hospital. Data communications between components of the door monitor system 100 related to the monitoring of a door are shown schematically by arrows.

Door monitoring system 100 comprises one or more doors. A single door 102 is shown in FIG. 1 for simplicity. These doors may be, for example, the doors to patients’ rooms in a hospital. It is important to monitor these doors to ensure patient welfare. Each door 102 may comprise one or more patient safety devices 104a-c. As noted above, patient safety devices 104a-c are devices included in the door (whether the door leaf or frame) that are configured to mitigate self-harm and/or aid patient welfare in some way. Example patient safety devices 104a-c and additional features of each door 102 are described in more detail in relation to FIG. 2. The door 102 of FIG. 1 has three patient safety devices 104a-c, however there may be any suitable number of these devices. Each patient safety device 104a-c may be configured to enable monitoring of a different respective aspect of the door 102 or surrounding environment.

As shown in FIG. 1, each door 102 also comprises an embedded electronics module 106. The embedded electronics module 106 is configured to receive status data from one or more patient safety devices 104a-c and transmit the status data to a computer apparatus 108 of the door monitoring system 100. Computer apparatus 108 is configured to perform the methods described herein, namely by processing and responding to the received patient safety device status data. The computer apparatus 108 may, for example, comprise a controller of the door monitoring system 100 having a processor and memory that is configured to run a suitable software application for implementing the disclosed methods. An example implementation of computer apparatus 108 is described below with reference to FIG. 6. It will be appreciated that there may be more than one computer apparatus 108 on the network and that the plurality of computer apparatuses may jointly implement the disclosed methods. For simplicity, only a single computer apparatus 108 is shown in FIG. 1.

In order to facilitate understanding, the door 102, patient safety devices 104a-c and embedded electronics module 106 will now be described in more detail with reference to FIGS. 2 and 3, before the remaining components of FIG. 1 are described.

Advantageously, in the present disclosure the embedded electronics module 106 is configured to provide the status data received from patient safety devices 104a-c to computer apparatus 108 via one or more serial communications protocols. Example serial communications protocols that can be used will be apparent to the skilled person, and include USB, Ethernet and OneWire protocols amongst others. Use of one or more serial communications protocol to communicate with computer apparatus 108 means the embedded electronics module 106 is able to take in the multiple inputs from the various patient safety devices 104a-c and provide these via a single output port to the computer apparatus 108 of the door monitoring system 100. This is highly advantageous because it means that there is no need to provide multiple parallel connections between computer apparatus 108 and each patient safety device 104a-c, which would be difficult and require a large amount of wiring and connections to be set up in implementations where each door 102 has several patient safety devices 104a-c.

This pooling or concentration of status data via the embedded electronics module 106 is shown more clearly in FIG. 2, which shows a schematic representation of a door 202 according to an example of the present disclosure. Door 202 corresponds to door 102 of FIG. 1 and comprises door leaf 203 and hinge 204 for hanging the door leaf 203 in a frame of the door. A handle 206 enables the door to be opened. The door frame is not shown in FIG. 2, for simplicity, however one or more patient safety devices 104a-c and/or the embedded electronics module 106 may be provided within the door frame in some implementations.

The door 202 of FIG. 2 comprises a variety of patient safety devices corresponding to patient safety devices 104a-c of FIG. 1. In particular, in this example door 202 comprises a ligature-detection device 208, a door observation panel 210, a shock sensor 212, an anti-barricade switch 214 and a door-open monitor 216. Each of these patient safety devices is configured to mitigate self-harm by an occupant of the room and/or facilitate wellbeing of the occupant by making it easier for staff to administer assistance. It will be appreciated that any combination of these patient safety devices may be provided in a door 202, and additional patient safety devices not shown in this example may also be provided.

To facilitate understanding, each patient safety device mentioned above will now be described in further detail.

Turning first to ligature-detection device 208, this is a device configured to detect if a ligature has been attached, or is in the process of being attached, to the door leaf 203. A ligature in this context means a rope, cable, noose or other binding which may be used by a person to harm themselves once secured to a door. The systems of the present disclosure can record when the status of ligature-detection device 208 changes, for example when ligature-detection device 208 is triggered.

A variety of ligature-detection devices are available and can be used in the context of the present disclosure. For example, United Kingdom patent applications GB1916899.6 and GB1918884.6, both in the name of Kingsway Enterprises (UK) Limited, disclose example ligature-detection devices which use ribbon switches to detect pressure applied by a potential ligature secured to the door. The disclosures of these references are hereby incorporated in full. Similarly, GB2109187.1, also in the name of Kingsway Enterprises (UK) Limited, discloses an inductive ligature-detection device which uses inductance to detect a potential ligature. The disclosure of this reference is also hereby incorporated in full. Other examples of ligature-detection devices will be known to the skilled person and can be used in the systems and methods of the present disclosure.

Turning to the next patient safety device, door observation panel 210 is a panel configured to provide a viewing window through which staff can monitor the occupant of a room for safety, security and wellbeing reasons. For reasons relating to privacy and occupant comfort, the viewing window is typically closed for the majority of the time and only opened when staff perform a check on the occupant. Door observation panel 210 can be opened in a number of ways, for example manually using a thumb-turn or handle or electronically by using a button. The systems of the present disclosure can record when the status of door observation panel 210 changes, for example when door observation panel 210 is opened. This is useful for ensuring that patients are being checked in on frequently by staff members, which ensures quality and consistency of care and patient safety.

Shock sensor 212 is a device configured to detect impacts against the door 202, for example as a result of a patient being violent (e.g. hitting or kicking the door) or attempting self-harm against the door. Any suitable sensor for identifying percussive shocks of this nature can be used as shock sensor 212 and the details thereof will be apparent to a skilled reader. The systems of the present disclosure can record when the status of shock sensor 212 changes, for example when shock sensor 212 records an impact against door 202. A threshold may be set to avoid false-alarms, with shocks above the threshold being identified as a status change of the shock sensor 212 and shocks below the threshold being ignored.

Anti-barricade switch 214 is a device configured to enable the door leaf 203 to be opened outwards. This is useful in scenarios where the occupant of the room has barricaded the door such that the door leaf 203 can no longer swing into the patient’s room. By triggering anti-barricade switch 214, the door leaf can be opened outward (away from the room) to enable staff access. The systems of the present disclosure can record when the status of anti-barricade switch 214 changes, for example when anti-barricade switch 214 is triggered.

Turning to the final patient safety device in the example of FIG. 2, door-open monitor 216 is a device configured to determine when door 202 has been opened. A variety of suitable sensors, such as pressure or inductive sensors, can be used and the details thereof will be apparent to a skilled reader. Door-open monitor 216 may identify, for example, when door leaf 203 is moved away from the door frame, and/or when handle 206 is operated to open the door. The systems of the present disclosure can record when the status of door-open monitor 216 changes, for example when door 202 is opened.

One option for obtaining the status data from the patient safety devices 208-216 of FIG. 2 would be to have a parallel connection from each patient safety device to computer apparatus 108 of the door monitoring system 100. However, the present inventors have identified that this arrangement would require a significant amount of cabling, with a separate cable running from the computer apparatus 108 to each patient safety device 208-216. This would make configuring and building the monitored door more difficult, and the increased number of components would mean that maintenance and repair is required more frequently and is more involved. If an intermediary component, such as a network bridge, were used then the number of patient safety devices that can be monitored may be limited by the number of input ports of the network bridge. As a result, such a system would limit the number of patient safety devices that could be implemented.

Accordingly, the inventors of the present disclosure have provided an improved system which seek to address these potential limitations. As already noted in relation to FIG. 1, the systems and methods of the present disclosure provide an embedded electronics module 106. This embedded electronics module 106 is designed to provide a single point of contact for door 202, from which computer apparatus 108 can obtain status data from a plurality, and preferably all, patient safety devices of the door 202. As shown in FIG. 2 by way of dashed arrows, embedded electronics module 106 obtains status data from each of patient safety devices 208-216. Computer apparatus 108 can then obtain all of the relevant status data via a single connection from embedded electronics module 106. Advantageously, the connection between computer apparatus 108 and embedded electronics module 106 is via a serial communications protocol as noted above. This provides an efficient, fast and reliable means for the computer apparatus 108 to correspond with embedded electronics module 106 and obtain the status data.

Embedded electronics module 106 is shown schematically in FIG. 3. As shown, embedded electronics module 106 has a number of inputs 302a-c. Three inputs are shown, but it will be appreciated that there may be more or fewer to accommodate the number of patient safety devices being monitored. There may be one input per patient safety device. Embedded electronics module 106 also has an output 304 which comprises a serial communications port suitable for communication with the computer apparatus 108. For example, output 304 may be a USB, Ethernet or OneWire port, or another similar serial communications port. Embedded electronics module 106 therefore effectively concentrates a number of input signals, received via input ports 302a-c, into a single output signal, provided at output port 304.

Returning now to the remaining features of the door monitoring system of FIG. 1, in this example door monitoring system 100 further comprises one or more peripheral devices 110. These may be other devices provided in the same building or area as monitored door 102, the statuses of which are to be monitored and recorded by computer apparatus 108. Status changes for peripheral devices 110 can be monitored and responded to in a similar manner as the status changes of patient safety devices 104a-c.

Examples of peripheral devices 110 include staff attack alarms. Staff working in psychiatric wards often carry such devices which may comprise fobs or devices comprising a button or trigger which can be actuated in case of an attack by a patient. Additionally or alternatively, hardwired staff alarm devices may be provided at fixed locations within the building. When these staff alarms are triggered, such a trigger can be treated as a status change and logged and recorded by door monitoring system 100 in the same way as status change data coming from the patient safety devices of a door 102. Other examples of peripheral devices that can be monitored in this way include patient call devices. For example, a patient in a bedroom may depress a button on a patient call device to call for assistance. Such an event can similarly be recorded as a status change by the door monitoring system 100.

Other examples of peripheral devices 110 include wearables worn by staff and/or patients. Such wearables can be in the form of existing wearable technology (e.g. a smartwatch) or a bespoke wearable (e.g. wrist band) used by the institution where the monitoring system is provided. Such wearables, as well as staff attack alarms, can enable geo-location tracking (e.g. using Bluetooth®, GPS or other similar technology to enable real-time or periodic location tracking) for tracking the location of staff and/or patients within the facility. Wearables can also be used to monitor vital signs (e.g. heartrate) for monitoring purposes. This is particularly useful for monitoring the health and wellbeing of, for example, patients in a psychiatric ward. It is important to monitor the vital signs of such patients frequently, however it may be undesirable to require staff to manually check the patients multiple times per day. A wearable provides a less intrusive mechanism for monitoring the wellbeing of the patients. In some cases, an unexpected or suspicious change in occupant vital signs can also be identified and treated as a status change by the door monitoring system 100. Other vital sign monitors such as bedsheets incorporating sensors (e.g. vibration sensors) can also be used. Such devices enable patients to be monitored at all times, including at night, without disturbing them.

Returning again to FIG. 1, door monitoring system 100 further comprises a display 112 connected to computer apparatus 108. Computer apparatus 108 may be configured in some implementations to use display 112 to visually inform staff of a change of status at a patient safety device 104a-c of a monitored door 102. Display 112 may be provided, for example, in a staff room or in a corridor of the building in which doors are being monitored, such that there is a high likelihood that a member of staff is nearby to the screen at all times in order to respond to displayed alerts and alarms. In some examples, use of display 112 may advantageously involve displaying a floor plan of a building comprising the monitored door 102 and indicating that a status of the patient safety device 104a-c has changed on the displayed floor plan. For example, the appearance of the monitored door 102 on the displayed floor plan may be modified in response to a status change. Similar functionality can be used to display, on the displayed floor plan, information about a status change detected at a peripheral device 110. An example implementation of a displayed floor plan that can be used in this manner is described in further detail below in relation to FIG. 5.

The final component of the door monitoring system 100 shown in FIG. 1 comprises alarm system 114. Alarm system 114 comprises one or more alarms, such as sirens and/or warning lights, situated around the building in which door 102 is located. Under control of computer apparatus 108, one or more alarms of alarm system 114 may be triggered in response to a status change of a patient safety device 104a-c or a peripheral device 110. It will be appreciated that alarm system 114 and display 112 complement one another and increase the likelihood that staff are alerted to high risk events. That said, in some cases one of the display 112 or alarm system 114 may be omitted and the system may rely on only the visual display or the alarms to draw staff attention.

Turning now to FIG. 4, an example method of using the disclosed door monitoring system to monitor a door is disclosed. The method may be performed by a controller comprising computer apparatus 108 of FIG. 1.

The method begins, at step 402, by monitoring a door. This step may involve a controller of door monitoring system 100 being configured to receive status data associated with the door in question. In particular, computer apparatus 108 may be connected to embedded electronics module 106 via one or more serial communications protocols. The serial communications protocol connection is preferably hardwired from end-to-end. In other words, a hardwired cable-based serial communications protocol connection carries the status data all the way from embedded electronics module 106 to computer apparatus 108. This may be over an Ethernet, USB, or OneWire cable, for example, or a combination of multiple such cables or other serial communications protocols.

Providing door monitoring system 100 as a hardwired system in this manner provides numerous benefits. In particular, such a connection is more reliable than a wireless connection. For example, a wireless connection is vulnerable to drop-out or to the use of Wi-Fi blockers or similar technology. Wireless devices are typically also battery powered, meaning they may fail if batteries are not replaced regularly. All of these factors increase the risk that a door monitoring system, particularly patient safety devices, may be rendered inoperable at some point, placing patients at risk because changes of status (for example of a ligature-detection device) may not be recorded or received by the door monitoring system. By using a hardwired connection instead, these risks are avoided. Use of a hardwired connection also provides a more secure connection for sensitive data, as may often be transferred in settings such as hospitals. In some implementations, power is carried over the one or more serial communications connections to power the embedded electronics module 106, patient safety devices 104a-c and/or any other intermediary devices. This further simplifies the cabling and architecture required.

At step 404, the method comprises receiving status data from the embedded electronics module 106 via the one or more hardwired serial communications protocols. As explained above, this status data can comprise data for a plurality of patient safety devices 104a-c, for example devices 208-216 of FIG. 2. Status data may be received at step 404 in response to a direct message sent by the patient safety device 104a-c responsive to a status change, said message then transmitted via the embedded electronics module 106 to computer apparatus 108. Alternatively or additionally, the embedded electronics module 106 may periodically (e.g. hundreds of times per second) poll the patient safety device 104a-c to check for a status change and/or to ensure that patient safety devices 104a-c are operational and responsive. Similarly, computer apparatus 108 may periodically (e.g. every 10 seconds) poll embedded electronics module 106 for the status change data and/or to ensure that embedded electronics module 106 is operational and responsive. In some cases, embedded electronics module 106 transmits data indicative of a change of status to the computer apparatus 108 immediately, without waiting to be polled.

At step 406, the method comprises determining, based on the received status data, that a status of a patient safety device 104a-c has changed. For example, as described above in relation to FIG. 2, computer apparatus 108 may determine that ligature-detection device 208 has been triggered, or that shock sensor 212 has detected a shock.

Additionally, embedded electronics module 106 may be configured to monitor and transmit additional forms of status data, and this can be reflected in the status data received at step 404 and the resulting status changes identified at step 406. For example, a door may comprise devices or sensors for determining if a fault has occurred at one or more patient safety devices 104a-c. Alternatively or additionally, lack of response by a patient safety device 104a-c to a polling or status update request message from embedded electronics module 106 may be taken as indicative of a fault with that patient safety device 104a-c. Regardless of how a fault is identified, embedded electronics module 106 may transmit data indicative of the fault at patient safety device 104a-c to computer apparatus 108. Based on the data, the door monitoring system 100 may thereby determine, at step 406, that a fault with one or more patient safety devices has been detected. Additionally, door monitoring system 100 may be configured to detect the disconnection of any patient safety devices 104a-c, embedded electronics module 106, or intermediary devices or computer apparatuses. Powered devices may be configured to report loss of power, either by a signal line or by a failure to respond to a status update request transmitted by computer apparatus 108. Any such events or changes in state can be received as status data at step 404 and the resulting status change identified therefrom at step 406. For example, the received data may indicate that the status of a patient safety device 104a-c can no longer be accurately determined, and/or that a patient safety device 104a-c has not responded to a status update request.

In addition to the data received at step 404 from the embedded electronics module 106, computer apparatus 108 may also receive status data from one or more peripheral devices 110 as described above. Similar status changes can be determined for the peripheral devices 110 based on this received data.

If no status changes are identified at step 406, monitoring continues and the method returns to step 402. If a status change is identified, then at step 408 the method comprises recording the status change. For example, a status change may be stored alongside suitable date, time and location data in a database for future auditing. This provides an electronic, reliable record of status changes that is not reliant on staff manually recording data and is also tamper-resistant. This reliable log of status change events thereby helps to ensure patient safety and quality of care. The data can be recorded to a local or remote database.

At optional step 410, identified status changes can also be displayed on a displayed floor plan. This floor plan may, for example, be displayed at display 112 of FIG. 1. In particular, in certain advantageous implementations the method may comprise displaying a floor plan of a building comprising the monitored door and indicating that a status of a patient safety device of the door has changed by modifying the appearance of the monitored door on the displayed floor plan. Status changes associated with peripheral devices 110 can also be displayed on the same floor plan. To aid understanding of this functionality, an example GUI that can be used to implement step 410 will now be described with reference to FIG. 5.

FIG. 5 shows a graphical user interface (GUI) 500 of the sort which can be displayed by computer apparatus 108 to implement step 410 of the method of FIG. 4. Specifically, this interface can be displayed on one or more displays or screens connected to computer apparatus 108, such as display 112 of FIG. 1. It will be appreciated that multiple displays each showing GUI 500 may be provided and situated at appropriate locations throughout the building being monitored. GUI 500 shows a floor plan 502 of the building in which the door monitoring system 100 is provided. As can be seen, floor plan 502 shows the rooms of the building and any associated doors.

During a setup phase, GUI 500 may be configured such the appearance of a monitored door is modified in response to the door monitoring system 100 identifying a status change at that door, such as at step 406 of the method of FIG. 4. For example, in one implementation, an icon may be provided in GUI 500 that is linked to the status data for a particular door. If a status change is detected, the appearance of the icon associated with that door changes. During the setup phase, the icons may be moved to the appropriate location on the floor plan 502 such that they overlay the door to which they relate. This can be accomplished, for example, using a “drag-and-drop” functionality whereby a user drags the icon to the correct location on the floor plan 502.

In the example of FIG. 5, four doors have been selected for monitoring and each door has as associated icon 504 which has been placed over the respective door. It will be appreciated that icons 504 can in practice take any suitable form and may be coloured and/or dynamic. Icons associated with other devices that are being monitored, such as peripheral devices 110, may also be displayed on floor plan 502. For devices that are mobile, such as staff attack alarms or wearables, GPS or other geolocation data may locate the device and be used to ensure that the associated icon 504 on floor plan 502 moves around to show the current location of the device in question.

Once configured in this manner, GUI 500 can be used to intuitively indicate to staff in a hospital or psychiatric ward where a new status change has occurred. Consider, for example, a trigger event at a ligature-detection device, such as device 208 of FIG. 2, occurring at one of the monitored doors. This may be indicative that a person is attempting to secure a ligature to the door in question. In response to identifying that a status of the patient safety device has changed, computer apparatus 108 is configured to modify the manner in which the door to which this device belongs is presented on the GUI 500. For example, the icon 504 provided at the door in question may begin to flash vigorously and/or change colour. By looking at the GUI, staff can thus immediately determine where the trigger event is occurring, because the icon 504 associated with the door in question has changed in appearance. In this way, staff are provided with an immediate and intuitive understanding of where in the building assistance is required.

Different visual modifications may advantageously be associated with different event types and status changes (e.g. door opening, fault detected, ligature trigger). In this way, staff can determine at a glance the event type and severity of a status change. This can enable staff to triage and attend the most severe events first, in case more than one event happens simultaneously. As can be seen, therefore, GUI 500 provides an intuitive and effective means of quickly alerting staff as to the nature and location of an event or status change in the building being monitored.

A fault list 506 can also be provided and shows faults that have recently been identified by the door monitoring system 100 and is displayed as part of the GUI 500. This provides an easily understandable indication of what faults have occurred at which doors or devices, with technical detail provided to enable staff to determine a remedy. For example, if a patient safety device of a door (such as one of devices 208-216 of FIG. 2) has not responded to a recent status update request transmitted by computer apparatus 108, then this may identify a fault with said device. This fault can be recorded in fault list 506. Similarly, recent status changes can be recorded and displayed in another list 508. In combination with the graphically displayed floor plan 502, fault list 506 and alarm list 508 provide staff at-a-glance with all the information required to respond to an event quickly and effectively.

Ward activity, including faults, status changes and potentially other data may also be consolidated and listed in a ward activity feed 510. Data from fault list 506, alarm list 508 and/or ward activity feed 510 can be recorded to a local or remote database by computer apparatus 108. As noted above, this advantageously means that a single system (door monitoring system 100) is used to detect events, alert staff, and record event history logs. No interfacing between multiple third party systems is required, and event log data is available from the door monitoring system 100 directly. This avoids the need to interface with any third party systems, thereby improving reliability and auditability.

Returning now to the method of FIG. 4, at optional step 412 the door monitoring system 100 may activate one or more alarms such as those of alarm system 114 of FIG. 1. This may happen simultaneously to the modification of the displayed floor plan just described in relation to step 410 of FIG. 4.

The visual modification performed at step 410 and/or the alarm generated at step 412 may advantageously differ based on which patient safety device 104a-c or peripheral device 110 has recorded a status change. This informs staff immediately of the severity of the event. More specifically, the visual modification and/or alarm can be based on a risk level associated with the status change identified at step 406. These risk levels associated with certain status changes can be configured or pre-programmed during setup or configuration of the door monitoring system.

For example, opening or closing of a door may be deemed a low-risk event. Hence, when such a status change is identified, an icon associated with the opened door may flash green and no auditory alarm may be sounded. A fault with a ligature-detection device or other patient safety device (or non-response therefrom) may be considered a medium-risk event. Hence, an icon associated with a door having such a fault may flash yellow and a brief auditory alarm may be sounded. Triggering of a ligature-detection device may be considered a high-risk event. Hence, an icon associated with a door having such a status change may flash red and expand, and a persistent auditory alarm may be sounded. It will be appreciated that these are merely examples of how visual and auditory cues can be used to intuitively communicate a door status change to staff. Many modifications and variations of this scheme will be apparent to a skilled reader and can be implemented within the disclosed systems and methods.

In addition to the above-described functionality, in some implementations the disclosed door monitoring system 100 can advantageously differentiate between potential false alarms and genuine status changes requiring a response. This is performed by applying a thresholding process to the determination of a status change performed at step 406.

This functionality is intended to address the fact that some patient safety devices 104a-c or peripheral devices may be accidentally triggered through benign actions. For example, a patient or staff member may brush against or bump into the edge of a door leaf when entering or exiting a room, thereby triggering the ligature-detection device 208 and/or shock sensor 212. Pressure may also be applied to the door edges during routine cleaning, for example, or through a variety of benign interactions with a door that are not associated with the attempt to attach a ligature.

To avoid false alarms being generated by events such as these, in some cases status changes that last for less than a minimum threshold time are ignored by the system, i.e. are not recorded as status changes at step 408. For example, in some cases status changes lasting less than seven seconds are ignored. If the status changes lasts for longer than seven seconds, then they are treated as a status change and are recorded as such at step 408. Alternatively, in some cases all status changes, however brief, are recorded at step 408 but only status changes lasting more than a threshold duration cause a modification at the displayed floor plan (at step 410) or cause an alarm to be activated (at step 412).

In some implementations, status changes below a threshold (e.g. seven seconds) result in a first type of visual modification (at step 410) and/or alarm (at step 412), referred to as a “pre-alarm”. Then, if the status change persists longer than the threshold, a second form of visual modification (step 410) and/or alarm (step 412) is generated. This functionality solves the above-described false alarm problem without compromising patient safety. In particular, the system enables brief, likely innocuous trigger events to be easily and intuitively distinguished from longer, potentially more serious trigger events. This functionality avoids the scenario where every slight trigger of the ligature-detection device results in a full-blown alarm being sounded. However, because pre-alarm events still result in some minor form of notification, staff are less likely to be taken unawares by a subsequent full alarm. As a result, an effective balance between avoiding false alarms and ensuring patient safety can be achieved.

The benefit of the disclosed approach may be further understood through consideration of the following illustrative examples. In a first scenario, a patient entering their room in a psychiatric ward touches the edge of their room door in an entirely benign manner. This is detected by the ligature-detection device 208 on the door. As a result, a pre-alarm is sounded (via an audio indication) and a first visual modification is generated on a display showing the monitored door. Because only a pre-alarm has been sounded, no response is yet required and resources are not wasted on investigating an innocuous detection event. In this example, no further triggers at the door in question are detected and so no further alarms are sounded. Staff members can continue to monitor the facility as before.

In a second scenario, a patient in their room in a psychiatric ward begins attempting to attach a ligature to their door. This will generally require touching the edges of the door several times as the person attempts to attach the ligature securely. Each touch, even if short, will result in a pre-alarm. While one pre-alarm may not be suspicious, several pre-alarms in short succession are. As a result, staff at the facility are forewarned of a potential ligature attachment event. Staff can similarly be forewarned if a patient is tampering with the ligature-detection system, which will similarly be characterised by several detection events in quick succession. This pre-alarm functionality thus enables pre-emptive investigation of the room in question. If the succession of pre-alarms is then followed shortly after by a full alarm, then staff have good reason to believe that the event that has been detected is indeed a real ligature-attachment event. Because staff have been alerted to this in a systematic and intuitive manner, they are able to administer assistance more rapidly than if no pre-alarm had been sounded prior to the full alarm.

As can be seen, the disclosed functionality enables reduced false alarms, enables innocuous touch events to be distinguished easily and intuitively from more suspicious events, and enables staff members to be forewarned of potential ligature attachment events in a way that would not be possible if short-duration events were simply ignored.

Even if pre-alarms are not associated with any auditory or visual indication, the differentiation between pre- and full alarms can still be useful. For example, the system may monitor how many pre-alarm events occur at a given door in a particular time-frame. If more than a threshold number of pre-alarm events occur in the time-frame, then this may be deemed suspicious behaviour and the system can notify a user (e.g. supervisor) via a display, for example in a notification or by drawing the user’s attention to the door in question via a visual modification or alert.

The present inventors have identified that the threshold duration for delimitating between pre- and full- alarms should advantageously be 2 seconds or more. Most benign triggerings of patient safety devices last less than 2 seconds, and so having a threshold duration of 2 seconds or more ensures that false alarms are effectively avoided.

The present inventors have also identified that the threshold duration for delimitating between pre and full alarms should advantageously be 7 seconds or less. If the threshold is higher than 7 seconds, then the system may be too slow to trigger a full alarm in response to an actual status change at a patient safety device that may represent a high risk situation.

Hence, advantageously the threshold duration which defines the boundary between a pre- and full- alarm is between 2 and 7 seconds inclusive. The present inventors have identified that a threshold of 7 seconds is particularly suitable, ensuring an effective balance between avoiding false alarms and ensuring patient or inmate safety.

In summary, as will be apparent from the above disclosure, the disclosed systems and methods provide a door monitoring system capable of monitoring various types of information and recording all data though a single streamlined system. As a result, the disclosed systems and methods can provide contextually rich information for a variety of events that can occur in a psychiatric ward or other similar environment. Staff can access all of this data through a single system, improving usability and avoiding confusion. The disclosed implementations can also provide a single record or database for auditing purposes which means there is no need to audit or interface with several different systems. The use of serial communications protocol makes the system and data architecture more efficient and reliable.

Turning finally to FIG. 6, FIG. 6 shows a schematic and simplified representation of a computer apparatus 600 which can be used to perform the methods described herein, either alone, in combination with other computer apparatuses or as part of a “cloud” computing arrangement. In particular, computer apparatus 600 may correspond to, or provide the functionality discussed in relation to, computer apparatus 108 discussed above with reference to FIGS. 1-5.

The computer apparatus 600 comprises various data processing resources such as a processor 602 (in particular a hardware processor) coupled to a central bus structure. Also connected to the bus structure are further data processing resources such as memory 604. A display adapter 606 connects a display device 608 to the bus structure. Display device 608 may correspond to display 112 of FIG. 1. One or more user-input device adapters 610 connect a user-input device 612, such as a keyboard and/or a mouse to the bus structure. One or more communications adapters 614 are also connected to the bus structure to provide connections to other computer systems 600 and other networks, in particular the other components and network devices of the door monitoring system 100.

In operation, the processor 602 of computer apparatus 600 executes a computer program comprising computer-executable instructions that may be stored in memory 604. When executed, the computer-executable instructions may cause the computer system 600 to perform one or more of the methods described herein, in particular those described in relation to FIG. 4 above. The results of the processing performed may be displayed to a user via the display adapter 606 and display device 608. In particular, this may involve display of GUI 500 described in relation to FIG. 5. User inputs for controlling the operation of the computer apparatus 600, for example during configuration of the GUI 500, may be received via the user-input device adapters 610 from the user-input devices 612.

It will be apparent that some features of computer apparatus 600 shown in FIG. 6 may be absent in certain cases. For example, one or more of the plurality of computer apparatuses 600 may have no need for display adapter 606 or display device 608. This may be the case, for example, for particular server-side computer apparatuses 600 which are used only for their processing capabilities and do not need to display information to users. For example, computers configured to record event logs recorded by door monitoring system 100 may be of this nature. Similarly, user input device adapter 610 and user input device 612 may not be required in some cases. In its simplest form, computer apparatus 600 comprises processor 602 and memory 604.

The above detailed description describes a variety of exemplary arrangements of and methods of implementing and using a door monitoring system. However, the described arrangements and methods are merely exemplary, and it will be appreciated by a person skilled in the art that various modifications can be made without departing from the scope of the appended claims. Some of these modifications will now be briefly described, however this list of modifications is not to be considered as exhaustive, and other modifications will be apparent to a person skilled in the art.

Whilst the above description has focussed on settings in hospitals, in particularly psychiatric hospitals, it will be appreciated that the disclosed methods and systems will also find ready utility in many other settings where self-harm and inhabitant safety is required, such as prisons.

It will be appreciated that the patient safety devices 104a-c described above can comprise additional types of sensor to those described. Patient safety devices 102a-c may be more generally considered as, or may comprise, alarm trigger interfaces, configured to determine if an alarm or fault state has occurred at a door.

It will be appreciated that the networks and systems shown in FIGS. 1-3 and 6 are schematic and simplified. In reality, there may be numerous versions of the components shown as well as additional components. Some of the components, in particular one or more computer apparatus 108, may be provided off-site and can interface with the remaining components through a network. The network of components can be simple or of any complexity depending on the site requirements. For example, networks of door monitoring system 100 can extend to other sites and monitoring stations.

There may be one or more intermediary devices provided between the components of FIG. 1. For example, a microcontroller may be provided between embedded electronics module 106 and computer apparatus 108. As noted above, the system may also make use of more than one serial communications protocol. For example, the embedded electronics module 106 may transmit status data to the intermediary device (e.g. microcontroller) over a first serial communications protocol, such as OneWire. The intermediary device may then transmit the status data to computer apparatus 108 over a second serial communications protocol, such as TCP/IP. Different serial communications protocols have different ranges, and so use of more than one serial communications protocol can enable the door monitoring system 100 to adapt to buildings of different sizes. For example, the second serial communications protocol over which the intermediary device corresponds with the computer apparatus 108 may be better suited to long distance communications. This makes the system more suited for use in large buildings. It will be appreciated that any number of intermediary devices and/or serial communications protocols may be used as required. Accordingly, references to “a serial communications protocol” herein and in the appended claims should be interpreted as “one or more serial communications protocols”.

The GUI showcased in FIG. 5 is merely one example of how the presently disclosed methods can be implemented. It will be appreciated that any suitable floor plan design can be used. Similarly, any suitable icon or visual indicator for showing that a particular door is being monitored can be used, and these can be added to the GUI by the user in any suitable manner not limited to drag-and-drop. The position, style and size of all GUI elements shown is merely exemplary, as are the visual modifications discussed above which can be modified as required by each use case.

While various specific combinations of components and method steps have been described, these are merely examples. Components and method steps may be combined in any suitable arrangement or combination. This is particularly the case in the method of FIG. 4, where various steps (e.g. steps 408, 410, 412) can occur simultaneously or in a different order than shown. Components and method steps may also be omitted to leave any suitable combination of components or method steps falling within the scope of the appended claims.

While the focus of most of the examples and implementations described above focus on data received from patient safety devices 104a-c, it should be understood that data from peripheral devices 110 such as staff attack alarms can be processed in the same way. Hence, all functionality discussed in relation to the processing and subsequent treatment of status data from patient safety devices 104a-c applies equally to status data obtained from peripheral devices 110.

As noted in relation to FIG. 6, the described methods may be implemented using computer executable instructions. A computer program product or computer readable medium may comprise or store the computer executable instructions. The computer program product or computer readable medium may comprise a hard disk drive, a flash memory, a read-only memory (ROM), a CD, a DVD, a cache, a random-access memory (RAM) and/or any other storage media in which information is stored for any duration (e.g., for extended time periods, permanently, brief instances, for temporarily buffering, and/or for caching of the information). A computer program may comprise the computer executable instructions. The computer readable medium may be a tangible or non-transitory computer readable medium. The term “computer readable” encompasses “machine readable”.

The singular terms “a” and “an” should not be taken to mean “one and only one”. Rather, they should be taken to mean “at least one” or “one or more” unless stated otherwise. The word “comprising” and its derivatives including “comprises” and “comprise” include each of the stated features, but does not exclude the inclusion of one or more further features.

The above implementations have been described by way of example only, and the described implementations are to be considered in all respects only as illustrative and not restrictive. It will be appreciated that variations of the described implementations may be made without departing from the scope of the disclosure. It will also be apparent that there are many variations that have not been described, but that fall within the scope of the appended claims.

1. A computer-implemented method for performing by a controller of a door monitoring system, the method comprising:
monitoring a door, the door comprising one or more patient safety devices and an embedded electronics module configured to monitor a status of each patient safety device;
receiving status data from the embedded electronics module via a hardwired serial communications protocol;
determining, based on the received status data, that a status of one of the one or more patient safety devices has changed; and
recording the status change.

2. The computer-implemented method of claim 1,
wherein the one or more patient safety devices comprise a ligature-detection device, and wherein determining that a status of the ligature-detection device has changed comprises determining that the ligature-detection device has been triggered.

3. The computer-implemented method of claim 1,
wherein the one or more patient safety devices comprise a door observation panel, and wherein determining that a status of the door observation panel has changed comprises determining that the door observation panel has been opened.

4. The computer-implemented method of claim 1,
wherein the one or more patient safety devices comprise a shock sensor, and wherein determining that a status of the shock sensor has changed comprises determining that the shock sensor has recorded a shock impacting on the monitored door.

5. The computer-implemented method of claim 1,
wherein the one or more patient safety devices comprise an anti-barricade switch, and wherein determining that a status of the anti-barricade switch has changed comprises determining that the anti-barricade switch has been triggered.

6. The computer-implemented method of claim 1,
wherein the one or more patient safety devices comprise a door-open monitor, and wherein determining that a status of the door-open monitor has changed comprises determining that the door has been opened.

7. The computer-implemented method of claim 1, further comprising determining, based on the received status data, that a fault with one of the one or more patient safety devices has been detected.

8. The computer-implemented method of claim 1, further comprising determining, based on the received status data, that the status of one of the one or more patient safety devices can no longer be accurately determined.

9. The computer-implemented method of claim 1, further comprising determining, based on the received status data, that one of the one or more patient safety devices has not responded to a status update request.

10. The computer-implemented method of claim 1, further comprising:
displaying a floor plan of a building comprising the monitored door; and
indicating that a status of the patient safety device has changed by modifying an appearance of the monitored door on the displayed floor plan.

11. The computer-implemented method of claim 10, wherein modifying the appearance of the monitored door comprises one or more of:
visually emphasising the monitored door;
changing a colour associated with the monitored door;
changing the appearance of a visual indicator associated with the monitored door; and
adding the monitored door to a displayed list of doors for which status changes have been detected.

12. The computer-implemented method of claim 11, wherein modifying the appearance of the monitored door differs based on which patient safety device has recorded a status change.

13. The computer-implemented method of claim 1, further comprising:
displaying a floor plan of a building comprising the monitored door; and
indicating that a status of the patient safety device has changed by modifying, on the displayed floor plan, the appearance of a room or area adjacent to or associated with the monitored door.

14. The computer-implemented method of claim 1, wherein recording the status change comprises logging, in a database, an indication of the status change.

15. The computer-implemented method of claim 1, further comprising:
responsive to determining that a status of one of the one or more patient safety devices has changed, causing an auditory indication to be sounded.

16. The computer-implemented method of claim 15, wherein the auditory indication differs based on which patient safety device has recorded a status change.

17. The computer-implemented method of claim 1, further comprising periodically polling the embedded electronics module with a request for updated status data.

18. The computer-implemented method of claim 1, wherein the embedded electronics module periodically polls the one or more patient safety devices for an indication of their respective statuses.

19. A computer apparatus configured to perform the method of claim 1.

20. A computer-readable medium comprising instructions which, when executed by a computer, cause the computer to carry out the method of claim 1.

21. A computer program comprising instructions which, when the program is executed by a computer, cause the computer to carry out the method of claim 1.

22. A door monitoring system comprising a controller having a processor and memory, the controller configured to perform the method of claim 1.

23. A door for monitoring by a door monitoring system, the door comprising:
a door leaf;
one or more patient safety devices; and
an embedded electronics module configured to monitor a status of each patient safety device,
wherein the embedded electronics module is configured to transmit status data for the one or more patient safety devices to a controller of a door monitoring system via a hardwired serial communications protocol.

24. The door of claim 23, wherein the embedded electronics module is configured to receive input status data from a plurality of patient safety devices and provide the status data from the plurality of patient safety devices to the door monitoring system via a single serial communications protocol output port.
